(19) United States
(12) Patent Application Publication
(10) Pub. No.: US 2014/0141087 A1
(43) Pub. Date: May 22, 2014

(54) Oral Composition Comprising An Antacid, an Anaesthetic and an Inorganic Matrix Comprising Silicon Dioxide and Titanium Dioxide

(71) Applicant: Malvin Eutick, Northbridge (AU)

(72) Inventor: Malvin Eutick, Northbridge (AU)

(73) Assignee: Eupharma Pty Ltd, New South Wales (AU)

(21) Appl. No.: 14/127,682

(22) PCT Filed: Jul. 13, 2012
(86) PCT No.: PCT/AU2012/000845

§ 371 Date: Jan. 30, 2014

(30) Foreign Application Priority Data
Jul. 15, 2011 (AU) 2011902834

(51) Int. Cl. A61K 33/24; A61J 1/00; A61K 31/341; A61K 33/08; A61K 31/16; A61K 9/10

(52) U.S. Cl. CPC A61K 33/24; A61K 31/16; A61K 9/10; A61K 31/341; A61K 33/08; A61J 1/00
USPC 424/489; 514/616; 514/471; 424/617; 206/570

(57) ABSTRACT

The invention provides an oral composition comprising a pharmaceutically acceptable carrier, at least one antacid, an inorganic matrix comprising at least silicon dioxide and titanium dioxide, and an anaesthetic, wherein the composition is substantially evenly dispersed in the carrier and is palatable.

### FIELD OF THE INVENTION

This invention relates to compositions of gastric and/or oesophageal medicaments. In particular the invention relates to a composition including antacids which are well dispersed and palatable when included in a liquid formulation.

### BACKGROUND

A number of gastric and oesophageal diseases and disorders can cause severe discomfort, such as gas and flatulence, associated with acid digestion, hyperacidity, dyspepsia, gastritis, gastroesophageal reflux disease (GERD), oesophageal swallowing disorders, oesophagitis and Barrett's oesophagus, heartburn, and many other forms of gastritis, as well as the symptoms arising from the ulceration caused by gastric and/or oesophageal ulcers and from damage caused by ionising radiation used in the treatment of cancers of these organs. Decreasing acidity in the stomach for the symptomatic treatment of the pain and discomfort is most often effected by use of antacids. These drugs can also be used where there is ulceration or damage to the mucosal lining caused by a number of factors including radiation and cytotoxic drugs used in the treatment of cancer.

The primary cause of the symptoms of pain and discomfort is the low acidity or the high level of hydrogen ions in the gut. In more recent years drugs which interfere with the release of the hydrogen ion have been used for treatment. These include the H2 Receptors inhibitors such as cimetidine, ranitidine, famotidine and nizatidine, which competitively inhibit the histamine receptor, thereby suppressing gastrin stimulated acid secretion. Most recently, treatment has been by use of proton pump inhibitors such as omeprazole, esomeprazole, lansoprazole, pantoprazole, and rabeprazole due to their more rapid action and efficacy.

Nevertheless, a primary treatment, especially for short term therapy for pain and discomfort, is the use of antacids which neutralise gastric acid and reduce pepsin activity, which reduces as gastric pH rises above 4.0.

Antacids themselves can be generally divided into two classes: absorbable and non-absorbable. Examples of the absorbable class are sodium bicarbonate and calcium carbonate. These provide rapid and complete neutralization but may cause alkalosis and are usually used for short periods, generally no longer than 1 to 2 days.

The second class includes non-absorbable neutralizing salts such as magnesium hydroxide, aluminium hydroxide and bismuth salts. Also in this group are drugs such as sucralfate (a sucrose-aluminium complex) and alginates which act as coating agents preventing the stomach acid from reaching an inflamed area by forming a physical barrier over the region.

Some antacid formulations may contain mixtures of drugs including antifoaming agents such as simethacone as well as mixtures of the newer drugs aimed to reduce the amount of hydrogen ions released by the stomach mucosa. Such mixtures usually include one or more drugs known as the proton pump inhibitors or H2 Receptor antagonists.

Another useful mixture, especially for patients suffering stomach pain from the mucosal damage caused by radiation or cytotoxic drugs used in cancer treatment, is the admixture of an antacid with an acid stable anaesthetic such as oxethacaine.

Such antacid liquid preparations are unpleasant to take orally due to their “gritty” texture and “muddy” or “chalky” taste which is not disguised or masked by flavours or sweeteners. Further, since they contain large quantities of insoluble alkaline salts, the commercial mixtures often have a settling problem, which means they have to be well shaken prior to use. This is very difficult for older patients and children, and potentially leads to under-dosing of the antacid. This problem is compounded when the antacid formulation contains doses of other drugs such as the proton pump inhibitors or H2 receptor antagonists or an anaesthetic, which are then also dosed inaccurately.

New preparations such as chewable lozenges and chewable tablets and fast dissolving tablets have successfully masked these problems but there are no advantageous liquid formulations or powder formulations for making into suspensions which provide reasonable administration.

### SUMMARY OF THE INVENTION

In one form, although it need not be the only or indeed the broadest form, the invention resides in an oral composition comprising a pharmaceutically acceptable carrier, at least one antacid, an inorganic matrix comprising at least silicon dioxide and titanium dioxide, and an anaesthetic, wherein the composition is substantially evenly dispersed in the carrier and is palatable.

The composition can be a solution or a suspension.

In yet a further form of the invention, there is provided a kit for making the composition defined above, the kit comprising a first container including at least one antacid, an inorganic matrix comprising at least silicon dioxide and titanium dioxide, and an anaesthetic, and a second container including a pharmaceutically acceptable carrier, wherein combination of the contents of the two containers results in the substantially evenly dispersed and palatable composition.

In a further form, the invention provides a tablet or lozenge comprising at least one antacid, an inorganic matrix comprising at least silicon dioxide and titanium dioxide, and an anaesthetic, wherein the tablet or lozenge when taken orally is palatable.

In yet a further form, the invention provides a method of manufacturing an oral composition of the invention comprising mixing together at least one antacid, an inorganic matrix comprising at least silicon dioxide and titanium dioxide, and an anaesthetic, in a pharmaceutically acceptable carrier, wherein the composition is substantially evenly dispersed in the carrier and is palatable.

In all forms of the invention above, the antacid can be any appropriate antacid or combination of antacids, absorbable and/or non-absorbable. Preferably the antacid or combination of antacids is chosen from the group of non-absorbable neutralising salts such as hydroxides, bismuth salts, sucralfate, and alginates. Most preferably, the composition and suspension of the invention discussed above includes magnesium hydroxide and aluminium hydroxide. Preferably the magnesium hydroxide is included at a concentration of from about 0.5% to about 25% and the aluminium hydroxide at a concentration of from about 0.5% to about 25%.

In all the forms of the invention above, the anaesthetic is preferably an acid stable anaesthetic. The acid stable anaesthetic can be any appropriate anaesthetic or combination of anaesthetics. Preferably the anaesthetic is oxethacaine, also known as oxetacaine or oxethazaine.

The silicon dioxide can be included in the composition or suspension of the invention in any practicable concentration. Preferably silicon dioxide is included at a concentration from about 0.05% to about 5%, and more preferably from about 3% to about 5%. The silicon dioxide for use in the composition or suspension of the invention preferably has particle size from about 3 micron to about 8 micron, and more preferably about 5 micron. In the composition and suspension of the invention, the silicon dioxide is both a suspending agent and a taste and texture masking agent. The particle size of the silicon dioxide used in the invention enables the silicon dioxide to perform both these functions.

The titanium dioxide can be included in any appropriate concentration. Preferably, this agent is included in a range from about 0.01% to about 5% but preferably at from about 0.1% to about 1.0%.

In the composition of the invention, the combination of silicon dioxide and titanium dioxide form the inorganic matrix of the invention. Organic suspending agents are not used in the invention as the composition thereby avoids interaction of the matrix with organic drugs (including the anaesthetic) in the composition. This prevents absorption of the drug/s, interference with activity of the drug/s and instability issues which may arise with interaction between a matrix and the drug/s. In addition, when the composition of the invention includes an organic preservative (see below) such as a parabens, binding with a matrix can inactivate the preservative. This is avoided with the inorganic matrix of the invention.

The inorganic matrix of the invention imparts a pleasant taste and texture to a suspension of the invention. The matrix also keeps insoluble salts in suspension, when the composition is a suspension, and allows the salts to be easily re-suspended after non-use for any period. In particular, as the suspension is easy to resuspend, the dose taken by patients is uniform over a period of time, periodically dispensing from a single container of the suspension.

Where appropriate to the forms of the invention above, the pharmaceutically acceptable carrier can be any appropriate carrier or combination of carriers. Preferably the carrier is primarily water. However, other pharmaceutically acceptable liquids such as lipids, pharmaceutically acceptable oils, or mixtures thereof can be included.

Other agents can also be included in the composition of the invention, including flavouring agents, such as peppermint flavour. Together with the inorganic matrix of the invention, this provides for the patient a pleasant sense of ingesting a creamy mint sweet, or breath freshener. Patient compliance is thereby greatly enhanced.

The suspension of the invention also facilitates administration directly to the gut using nasogastric tubes if the throat of the patient does not allow swallowing.

The composition of the invention can also include other pharmaceutically appropriate stabilising and bioadhesive agents such as agar, alginate, carboxymethylcellulose, dextrates, pectin, hydroxypropyl-methylcellulose; and can also include lubricants and surfactants such as polyvinyl alcohol, castor oil or esters thereof, and polysorbates.

The composition of the invention can be formulated as a single dose, multidose, or can be provided in a kit as defined above, comprising a container, for example a sachet, of the composition, and a mixing container containing the carrier, optionally including additives as discussed above. A single dose can be provided in any practicable form, including as a pre-mixed sachet, on-site mixable kit, swallowable tablet, chewable tablet, effervescent tablet, lozenge, or ready-mixed suspension.

When the suspension of the invention is formulated as a multidose formulation, a pharmaceutically appropriate preservative or mixture of preservatives can be added to the suspension, such as benzyl alcohol, hydroxybenzoates (parabens) and ethanol.

The suspension or composition of the invention can optionally include, in addition to the acid neutralising salts or coatings, or anaesthetic drugs, the following classes of drugs for the purposes as indicated:
surfactants such as simethicone;
drugs (other than those salts used to treat hyperacidity, such as the hydroxides of magnesium and aluminium) that directly inhibit the release of hydrogen ions such as the proton pump inhibitors, for example: omeprazole or pantoprazole, esomeprazole, lansoprazole and rabeprazole and their derivatives; or the H2 receptor inhibitors such as ranitidine, cimetidine, nizatidine and famotidine and their derivatives;
drugs, and their combinations with or without a proton pump inhibitor, used to treat Helicobacter pylori infection: the causative agent of ulceration; such as bismuth salts, metronidazole, tetracyclines, clarithromycin and amoxicillin;
anti-inflammatory drugs such as benzydamine, ibuprofen, paracetamol, and non steroidal anti-inflammatory drugs (NSAIDs) and derivatives thereof;
drugs that may calm the patient such as anti-anxiolytics and sedatives, or that induce some conscious sedation of the patient such as midazolam and other benzodiazepines;
drugs that may reduce the nausea such as metoclopramide, ondansetron, granisetron, droperidol, and dexmedetomidine;
non-NSAID pain relieving drugs such as fentanyl and its derivatives, morphine, oxycodone, hydromorphone, nalbuphine and codeine;
sugars such as lactulose, galactose and lactose;
polyethylene glycol (macrogols) and glycerol;
other anti-acidic salts such as calcium carbonate, magnesium trisilicate, magnesium carbonate, sodium bicarbonate, sodium carbonate;
mucosal coating agents such as sucralfate (a sucrose-aluminium complex) and alginates;
carbon and paraffin and natural fibres and mucilloids such as psyllium and alginates, guar gum, and pectin;
prostaglandin derivatives such as misoprostol;
agents used for bowel preparation for radiography and colonoscopy such as sodium picosulphate, and phosphate and other salt mixtures such as mono and dibasic sodium phosphates, sodium sulphate;
biscodyl;
agents used to treat bowel diseases such ulcerative colitis or Crohn's disease and colitis such as mesalazine, sulfasalazine and balsalazine;
agents used to treat liver disease such as ursodeoxycholic acid and arginine hydrochloride.

Inclusion of any one of, or a combination of any of these drugs, into an oral suspension which improves patient compliance through its improved organoleptic qualities and better dosing via more uniform suspendability, is of great benefit for the treatment proposed. Dosing and patient compliance are improved, and therefore the chances of successful treatment or amelioration of symptoms is greatly enhanced.

Oral compositions and/or suspensions of the invention, with or without one or more of the additional drugs listed above, and/or the optional additives discussed earlier, provide a composition and method of administering an antacid and anaesthetic combination which is effective, safe and comfortable to the patient.

### DETAILED DESCRIPTION OF THE INVENTION

Detailed, non-limiting examples of the invention are provided.

### Example 1

### Water Based Antacid Suspension with Taste Masking

Per each 100 mL:
1.95 g magnesium hydroxide
6.12 g aluminium hydroxide
4.0 g silicon dioxide
0.5 g titanium dioxide
0.1 g methyl paraben
0.05 g propyl paraben
70% sorbitol to volume
0.5 mL peppermint oil

### Method of Preparation

### Part 1—Preparation of the Titanium Dioxide and Magnesium and Aluminium Hydroxides

1. Weigh out the magnesium hydroxide, aluminium hydroxide and the titanium dioxide.
2. Mix and blend and hammer mill or grind into a homogenous blend and bagged for mixing in the tank (keep dry).

### Part 2—Preparation of the Preservatives (Parabens)

1. Weigh out the two parabens.
2. Add to very warm pharmaceutical grade water (as low a volume as required) with stirring until dissolved.
3. Stir until fully dissolved (about 30 minute) and allow to cool.
4. Set aside in preparation for adding it to the main preparation.

### Part 3—Blending and Mixing of Ingredients

1. Add approximately 60 mL of 70% sorbitol solution to the dry hydroxides and titanium dioxide powder from Part 1 with stirring.
2. Slowly add the silicon dioxide.
3. Mix for 20 minutes.
4. Add the premixed parabens and stir.
5. Add peppermint oil and allow to cool.
6. Bring up to 100 mL volume with 70% Sorbitol solution and stir for 30 minutes.
NOTE: A colour may be added at the end of the final mixing step if necessary.

### Example 2

### Water Based Antacid Suspension with Taste Masking and an Anaesthetic

Per each 100 mL:
1.95 g magnesium hydroxide
6.12 g aluminium hydroxide
0.2 g oxethacaine base
4.0 g silicon dioxide
0.5 g titanium dioxide
0.1 g methyl paraben
0.05 g propyl paraben
70.0% sorbitol solution to volume
0.5 mL peppermint oil

### Method of Preparation

### Part 1—Preparation of the Titanium Dioxide and Magnesium and Aluminium Hydroxides

1. Weigh out the magnesium hydroxide, aluminium hydroxide and the titanium dioxide
2. Mix and blend and hammer mill or grind into a homogenous blend and bagged for mixing in the tank (keep dry).

### Part 2—Preparation of the Oxethacaine Base

1. Prepare a small volume of pharmaceutical grade water.
2. Adjust the pH with hydrochloric acid to approximately pH 3 (it can fall between 2.5-3.5) using 0.1M Hydrochloric acid.
3. Warm the solution but do not boil.
4. Add the oxethacaine base slowly with constant stirring the oxethacaine base.
5. Stir until fully dissolved and allow to cool.
6. Set aside in preparation for adding it to the main tank (no volume adjustment is needed).
7. Just prior to addition to the main tank, add a small amount of 0.1M NaOH to lift the ph above 5 and allow it to form a fine precipitate. This must be thoroughly re-stirred before addition.

### Part 3—Preparation of the Preservatives (Parabens)

1. Weigh out the two parabens.
2. Add to very warm pharmaceutical grade water (as low a volume as required) with stirring until dissolved.
3. Stir until fully dissolved (about 30 minute) and allow to cool.
4. Set aside in preparation for adding to the main preparation.

### Part 4—Blending and Mixing of Ingredients

1. Add approximately 60 mL of 70% Sorbitol solution to the dry hydroxides and titanium dioxide powder from Part 1 with stirring.
2. Slowly add the silicon dioxide.
3. Mix for 20 minutes.
4. Add the oxethacaine solution slowly with fast stirring to blend. It will form a fine precipitate on addition to the alkaline mixture and it is important that the blending is thorough.
5. Add the premixed parabens and stir.
6. Add peppermint oil and allow to cool.
7. Bring up to 100 mL volume with 70% Sorbitol solution and stir for 30 minutes.
NOTE: A colour may be added at the end of the final mixing step if necessary.

### Example 3

### Water Based Antacid Suspension with Taste Masking and a H2 Inhibitor (Ranitidine Hydrochloride)

Per each 100 mL:
1.95 g magnesium hydroxide
6.12 g aluminium hydroxide
3.0 g ranitidine hydrochloride
4.0 g silicon dioxide
0.5 g titanium dioxide
0.1 g methyl paraben
0.05 g propyl paraben
70.0% sorbitol solution
0.5 mL peppermint oil

### Method of Preparation:

### Part 1—Preparation of the Titanium Dioxide and Magnesium and Aluminium Hydroxides

1. Weigh out the magnesium hydroxide, aluminium hydroxide and the Titanium dioxide.
2. Mix and blend and hammer mill or grind into a homogenous blend and bagged for mixing in the tank (keep dry).

### Part 2—Preparation of the Ranitidine Hydrochloride

1. Prepare a small volume of pharmaceutical grade water.
2. Adjust the pH with hydrochloric acid to approximately pH 5 (it can fall between 4.5-6.5) using 0.1M Hydrochloric acid.
3. Warm the solution but do not boil.
4. Add the Ranitidine hydrochloride slowly with constant stirring.
5. Stir until fully dissolved and allow to cool.
6. Set aside in preparation for adding it to the main tank (no volume adjustment is needed).

### Part 3—Preparation of the Preservatives (Parabens)

1. Weigh out the two parabens.
2. Add to very warm pharmaceutical grade water (as low a volume as required) with stirring until dissolved.
3. Stir until fully dissolved (about 30 minute) and allow to cool.
4. Set aside in preparation for adding it to the main preparation.

### Part 4—Blending and Mixing of Ingredients

1. Add approximately 60 mL of 70% Sorbitol solution to the dry hydroxides and titanium dioxide powder from Part 1 with stirring
2. Slowly add the silicon dioxide.
3. Mix for 20 minutes.
4. Add the ranitidine hydrochloride solution slowly with fast stirring to blend.
6. Add the premixed parabens and stir.
7. Add peppermint oil and allow to cool.
8. Bring up to 100 mL volume with 70% Sorbitol solution and stir for 30 minutes.
NOTE: A colour may be added at the end of the final mixing step if necessary.

### Example 4

### Water Based Antacid Suspension with Taste Masking and an Anaesthetic (Oxethacaine) and a H2 Receptor Inhibitor (Ranitidine Hydrochloride)

Per each 100 mL:
1.95 g magnesium hydroxide
6.12 g aluminium hydroxide
0.2 g oxethacaine base
3.0 g ranitidine hydrochloride
4.0 g silicon dioxide
0.5 g titanium dioxide
0.1 g methyl paraben
0.05 g propyl paraben
70.0% sorbitol solution
0.5 mL peppermint oil

### Method of Preparation

### Part 1—Preparation of the Titanium Dioxide and Magnesium and Aluminium Hydroxides

1. Weigh out the magnesium hydroxide, aluminium hydroxide and the titanium dioxide
2. Mix and blend and hammer mill or grind into a homogenous blend and bagged for mixing in the tank (keep dry).

### Part 2—Preparation of the Oxethacaine Base

1. Prepare a small volume of pharmaceutical grade water.
2. Adjust the pH with hydrochloric acid to approximately pH 3 (it can fall between 2.5-3.5) using 0.1M Hydrochloric acid.
3. Warm the solution but do not boil.
4. Add the oxethacaine base slowly with constant stirring the oxethacaine base.
5. Stir until fully dissolved and allow to cool.
6. Set aside in preparation for adding it to the main tank (no volume adjustment is needed).
7. Just prior to addition to the main tank, add a small amount of 0.1M NaOH to lift the pH above 5 and allow it to form a fine precipitate. This must be thoroughly re-stirred before addition.

### Part 3—Preparation of the Ranitidine Hydrochloride

1. Prepare a small volume of pharmaceutical grade water.
2. Adjust the pH with hydrochloric acid to approximately pH 5 (it can fall between 4.5-6.5) using 0.1M Hydrochloric acid.
3. Warm the solution but do not boil.
4. Add the Ranitidine hydrochloride slowly with constant stirring.
5. Stir until fully dissolved and allow to cool.
6. Set aside in preparation for adding it to the main tank (no volume adjustment is needed).

### Part 4—Preparation of the Preservatives (Parabens)

1. Weigh out the two parabens.
2. Add to very warm pharmaceutical grade water (as low a volume as required) with stirring until dissolved.
3. Stir until fully dissolved (about 30 minute) and allow to cool.
4. Set aside in preparation for adding it to the main preparation.

### Part 5—Blending and Mixing of ingredients

1. Add approximately 60 mL of 70% Sorbitol solution to the dry hydroxides and Titanium dioxide powder from Part 1 with stirring.
2. Slowly add the silicon dioxide.
3. Mix for 20 minutes.
4. Add the ranitidine hydrochloride solution with stirring.
5. Add the oxethacaine solution slowly with fast stirring to blend. It will form a fine precipitate on addition to the alkaline mixture and its important that the blending is thorough.
6. Add the premixed parabens and stir.
7. Add Peppermint oil and allow to cool.
8. Bring up to 100 mL volume with 70% Sorbitol solution and stir for 30 minutes.
NOTE: A colour may be added at the end of the final mixing step if necessary.

Throughout the specification the aim has been to describe the preferred embodiments of the invention without limiting the invention to any one embodiment or specific collection of features.

Throughout this specification, unless the context requires otherwise, the word “comprises”, and variations such as “comprise” or “comprising”, will be understood to imply the inclusion of a stated integer or group of integers or steps but not to the exclusion of any other integer or group of integers.

1. An oral composition comprising a pharmaceutically acceptable carrier, at least one antacid, an inorganic matrix comprising at least silicon dioxide and titanium dioxide, and an anaesthetic, wherein the composition is substantially evenly dispersed in the carrier and is palatable.

2. The composition of claim 1 wherein the composition is a suspension.

3. The composition of claim 1 wherein the composition is a tablet or lozenge.

4. The composition of claim 1 wherein the antacid is selected from the group consisting of hydroxides, bismuth salts, sucralfate, alginates, and mixtures thereof.

5. The composition of claim 4 wherein the antacid is magnesium hydroxide, aluminium hydroxide or mixtures thereof.

6. The composition of claim 1 wherein the anaesthetic is one or more acid-stable anaesthetic.

7. The composition of claim 6 wherein the anaesthetic is oxethacaine.

8. The composition of claim 1 wherein the silicon dioxide has particle size from about 3 micron to about 8 micron.

9. The composition of claim 1 wherein the silicon dioxide is included in the composition at a concentration from about 0.05% to about 5%.

10. The composition of claim 1 wherein the titanium dioxide is included in the composition at a concentration from about 0.01% to about 5%.

11. The composition of claim 1 wherein the carrier includes water.

12. The composition of claim 1 further comprising one or more preservative.

13. The composition of claim 12 wherein the preservative is one or more paraben.

14. The composition of claim 1 further comprising one or more pharmaceutical other than anaesthetics.

15. A method of manufacturing an oral composition comprising mixing together at least one antacid, an inorganic matrix comprising at least silicon dioxide and titanium dioxide, and an anaesthetic, in a pharmaceutically acceptable carrier, wherein the composition is substantially evenly dispersed in the carrier and is palatable.

16. A kit for assembling the composition of claim 1, the kit comprising a first container including the at least one antacid, the inorganic matrix comprising at least silicon dioxide and titanium dioxide, and the anaesthetic, and a second container including the pharmaceutically acceptable carrier, wherein combination of the contents of the first and second containers results in the substantially evenly dispersed and palatable composition.
